(19) United States
(12) Patent Application Publication
(10) Pub. No.: US 2022/0378863 A1
(43) Pub. Date: Dec. 1, 2022

(54) METHOD AND COMPOSITION FOR REJUVENATION OF MUSCLE CELLS

(71) Applicant: NuLiv Science USA, Inc., Brea, CA (US)

(72) Inventors: Chia-Hua KUO, Taipei City (TW); Michael, Chang Yu WANG, Brea, CA (US)

(21) Appl. No.: 17/830,039

(22) Filed: Jun. 1, 2022

Related U.S. Application Data
(60) Provisional application No. 63/195,299, filed on Jun. 1, 2021.

(51) Int. Cl. A61K 36/258; A61K 31/351; A61K 31/375; A61K 31/05

(52) U.S. Cl. CPC A61K 36/258; A61K 31/351; A61K 31/375; A61K 31/05

(57) ABSTRACT

The present invention is related to a supplement for reversing the aging of muscle cells after an acute bout of squat exercise in a subject, which comprises, or consists essentially of, or consists of, (1) Panax ginseng extract or Panax notoginseng extract or a combination thereof and (2) Rosa roxburghii extract at an amount to reverse the aging of muscle cells in a subject after after an acute bout of squat exercise. Provided in the present invention are the method and use of the supplement Rg1 for reversing the aging of muscle cells in a subject after an acute bout of squat exercise.

### CROSS REFERENCE

This non-provisional application claims the priority under 35 U.S.C. § 119(a) on U.S. Patent Provisional Application No. 63/195,299 filed on Jun. 1, 2021, the entire contents of which are hereby incorporated by reference.

### FIELD OF THE INVENTION

The present invention pertains to a method and a composition for rejuvenating muscle cells in a subject.

### BACKGROUND OF THE INVENTION

Stem cell aging is featured by increased expression of the cell cycle inhibitor p16^{INK4a} in replicable cells [Boquoi et al., 2015]. This inhibitor halts cell regeneration, self-renewal and homing, resulting in stress intolerance and fitness decline at a higher age [Janzen et al., 2006; Justice et al., 2018]. Increasing p16^{INK4a} expression during cellular aging is also an intrinsic mechanism to lower mitotic error of aged stem cells [Campisi et al., 2005]. Since most of cells in human body are short-lived [Spalding et al., 2005], p16^{INK4a+} cells are widely detectable in embryonic [Storer et al., 2013] and young adult tissues [Yang et al., 2018; Ressler et al., 2006]. Lymphocyte p16^{INK4a} mRNA increases exponentially with age from 18 to 80 years old [Liu et al., 2009]. However, p16^{INK4a+} cell number is not different between young and old muscles measured by semi-quantitative immunohistochemical (IHC) analysis [Dungan et al., 2020]. This discrepancy suggests that the level of p16^{INK4a} mRNA is a more sensitive biomarker of tissue aging than p16^{INK4a+} cell number for humans.

The effect of resistance exercise on p16^{INK4a} mRNA in human skeletal muscle has not yet been reported. Based on IHC analysis, resistance exercise seems to suppress p16^{INK4a+} cell number surrounding myofibers in skeletal muscle of untrained men [Yang et al., 2018] and physically inactive women [Justice et al., 2018]. A significant proportion of p16^{INK4a+} cells are colocalized with CD34+ endothelial progenitor cells (EPC) adjacent to myofibers [Yang et al., 2018]. EPC contributes to reparative process of vascular endothelium [Zampetaki et al., 2008] and muscle regeneration in injured and ischemic tissues [Tamaki et al., 2002]. Vascular endothelial cells in muscle tissue age rapidly with a lifespan of around two weeks [Erben et al., 2008]. The levels of EPC aging and young EPC availability for cell regeneration directly influence the fitness of muscle tissues. Resistance exercise acutely mobilizes EPC from bone marrow into circulation until 6 h but quickly returned to baseline within 24 h of recovery [Ribeiro et al., 2017]. It remains unknown whether EPC is homing in human skeletal muscle during the rise in blood and expanding 24 h after an acute bout of resistance exercise.

Cellular senescence in tissues contributes to baseline inflammation [Lasry et al., 2015]. This is characterized by increased level of neutrophil infiltration in tissues, which can be detected by MPO mRNA level [Amanzada et al, 2011]. During inflammation, immune cells recognize and eliminate senescent cells to maintain youth of the tissue [Kay, 1975]. Accordingly, it is possible to find a solution to reverse the aging in human muscle through the stem cell aging model.

### SUMMARY OF THE INVENTION

It is unexpectedly found in the present invention that the aging of stem cells in human skeletal muscle after exercise were reversed through an administration of an Rg1 supplement comprising (1) Panax ginseng extract or Panax notoginseng extract or a combination thereof and (2) Rosa roxburghii extract.

One aspect of the invention is to provide am Rg1 supplement for use in rejuvenation of muscle cells in a subject, which comprises, or consists essentially of, or consists of, (1) Panax ginseng extract or Panax notoginseng extract or a combination thereof and (2) Rosa roxburghii extract at an amount to reverse the aging of muscle cells in the subject, wherein the Rg1 supplement is standardized to contain 30% to 40% of a total saponin, 0.6% to 2.0% of Vitamin C of, and 2.0%-4.0% of polyphenols, and a ginsenoside Rg1 as one indicator component ranging from 5 mg to 50 mg for one serving; and wherein the Rg1 supplement is administered to said subject after an acute bout of squat exercise.

Another aspect of the invention is to provide a method for rejuvenation of muscle cells in a subject, which comprises administering to said subject the Rg1 supplement after his/her completing of an acute bout of aerobic exercise.

One further aspect of the invention is to provide a use of the Rg1 supplement for manufacturing an active agent or a medicament for rejuvenation of muscle cells in a subject; and wherein the Rg1 supplement is administered to said subject after an acute bout of squat exercise.

In one example of the invention, the Rg1 supplement comprises, essentially consists of, or consisting of (1) Panax ginseng extract or Panax notoginseng extract or a combination thereof, and (2) Rosa roxburghii extract, at an amount to revise the aging of stem cells in a subject after an acute bout of squat exercise, wherein the Rg1 supplement is standardized to contain 30% to 40% (weight % in total) of a total saponin, 0.6% to 2.0% of Vitamin C, and 2.0% to 4.0% of polyphenols, and a ginsenoside Rg1 as one indicator component ranging from 5 mg to 50 mg for one serving; which is called as “Rg1 supplement” or “Rg1” hereinafter.

In the example of the invention, the Rg1 supplement enhances a reduction of stem cell aging in exercised skeletal muscle cells

In a particular example of the invention, the Rg1 supplement provides an efficacy in decreasing senescent cell markers and immune cell markers in exercised skeletal muscle, which was also confirmed by in vitro and animal studies. The example was undertaken to measure EPC number, p16^{INK4a} mRNA, and immune cell markers in human skeletal muscle 24 h following an acute bout of squat exercise.

The invention will be further described by way of the following examples. However, it should be understood that the following examples are solely intended for the purpose of illustration and should not be construed as limiting the disclosure in practice.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the plasma muscle damage markers unaltered following aerobic exercise, wherein lactate dehydrogenase (A), myoglobin (B), and TBARS (C) were not significantly increased after 1-h cycling exercise (70% VO2max). (Data are expressed as mean and SEM. Abbreviation: TBARS, Thiobarbituric acid reactive substances.)

FIG. 2 shows that the massive increases of senescent endothelial progenitor cells in human skeletal muscle 3 h after aerobic exercise (70% VO₂max). Approximately 21-fold increases in senescent endothelial progenitor cells occurred 3 h after 1-h cycling exercise (70% VO₂max), while the Rg1 supplementation advances the increase immediately after exercise (˜3-fold) and decline to baseline 3 h after exercise (A, C). Total endothelial progenitor cells (CD34+) surrounding myofibers were unaltered after exercise for both trials (B, D). Approximately 60% of elevated senescent endothelial progenitor cells was contributed by endothelial progenitor cells (p16^{INK4a+}/CD34⁺) (E, G), while the rest of 40% was contributed by infiltrated nucleated cells (p16^{INK4a+}/CD34⁻) (F, H). Scale bar=30 μm. (Data are expressed as mean and SEM. *Significant difference against Pre (Baseline), P<0.01; † Significant difference against Placebo, P<0.01. Abbreviation: PLA, Placebo.)

FIG. 3 shows the moderate correlation (r=0.29, p=0.08) between p16^{INK4a} positive cells and β-galactosidase positive cells of 36 biopsied vastus lateralis muscle in men aged 20-26 y. (Abbreviation: β-gal, β-galactosidase.)

FIG. 4 shows the aerobic exercise increases regenerative macrophage infiltration into human skeletal muscle; wherein the arrows in the representative images indicate CD163⁺ cells (bright green) and nuclei (blue) surrounding myofibers in a muscle cross-section (A). This increase after 1-h cycling exercise (70% VO₂max) was similar for both Placebo (PLA) and Rg1 trials (B). Scale bar=50 μm. (Data are expressed as mean and SEM. *Significant difference against Pre (Baseline), P<0.01. Abbreviation: PLA, Placebo.)

FIG. 5 shows the IL-10, VEGF, and PGC1-alpha expression after aerobic exercise; wherein (A) the Rg1 supplementation lowered IL-10 mRNA 3 h after exercise (−60%, P<0.05). Dramatic elevations for VEGF mRNA (B) and PGC1-alpha mRNA (C) occurred 3 h after 1-h cycling exercise (70% \dot{V}O2max). (Data are expressed as mean and SEM. *Significant difference against Pre (Baseline), P<0.05. Abbreviation: PLA, Placebo.)

FIG. 6 shows the biomarkers of cellular senescence in human skeletal muscle 24 h after squat exercise, wherein the p16^{INK4a+} cells (A) and β-Gal⁺ cells (B) were indicated by brown precipitates surrounding myofibers in the immunohistochemical stains of muscle cross-sections. No significant changes in quantity of p16^{INK4a+} cells (C) and β-Gal⁺ cells (D) were observed post exercise during PLA- and Rg1-administered trials. Squat exercise decreased p16^{INK4a} mRNA (E) while no effect on β-Gal mRNA (F) in vastus lateralis muscle of young men with training experience. (* denotes significant difference against Pre, p<0.05; ** denotes significant difference against Pre, p<0.01. Pre: before exercise; Post: 24 h after exercise. β-Gal⁺: β-Galactosidase; PLA: Placebo; Rg1: the Rg1 supplement.)

FIG. 7 shows the myeloperoxidase mRNA in human skeletal muscle 24 h after squat exercise. Myeloperoxidase mRNA (neutrophil marker) decreased post exercise when the Rg1 supplement was administered 1 h before exercise (A). Myeloperoxidase mRNA is highly correlated with p16^{INK4a} mRNA (B) in muscle tissues. (** denotes significant difference against Pre, p<0.01. Pre: before exercise; Post: 24 h after exercise. MPO: Myeloperoxidase; PLA: Placebo; Rg1: the Rg1 supplement.)

FIG. 8 shows the endothelial progenitor cell expansion (CD34⁺ cells) in human skeletal muscle 24 h after squat exercise; wherein the CD34⁺ cells and p16^{INK4a+} cells (B) were indicated by brown precipitates surrounding myofibers in immunohistochemical stains of muscle cross-section. (A) CD34⁺ cells were nearly doubled post exercise (C). Approximately 65% of CD34⁺ cells were colocalized with p16^{INK4a+} cells. This ratio was not much affected after exercise (60%) in the trained men. (* denotes significant difference against Pre, p<0.05; ** denotes significant difference against Pre, p<0.01. Pre: before exercise; Post: 24 h after exercise. PLA: Placebo; Rg1: the Rg1 supplement.)

FIG. 9 shows the CD4⁺ T lymphocyte infiltration in human skeletal muscle 24 h after squat exercise; wherein the CD4⁺ T lymphocytes and p16^{INK4a+} cells (A) were indicated by brown precipitates surrounding myofibers in immunohistochemical stains of muscle cross-section. CD4⁺ T lymphocytes were rarely detectable at baseline (Pre) and were increased significantly post exercise (B). Approximately 35% of CD4⁺ cells were colocalized with p16^{INK4a+} cells (C). (** denotes significant difference against Pre, p<0.01. Pre: before exercise; Post: 24 h after exercise. PLA: Placebo; Rg1: the Rg1 supplement.)

### DETAILED DESCRIPTION OF THE INVENTION

Unless defined otherwise, all technical and scientific terms used herein have the same meaning as commonly understood by a person skilled in the art to which this invention belongs.

As used herein, the singular forms “a”, “an”, and “the” include plural referents unless the context clearly dictates otherwise. Thus, for example, reference to “a sample” includes a plurality of such samples and equivalents thereof known to those skilled in the art.

It was confirmed in the present invention that the aging of stem cells in human skeletal muscle after exercise was reversed through the Rg1 supplement comprising (1) Panax ginseng extract or Panax notoginseng extract or a combination thereof and (2) Rosa roxburghii extract.

Accordingly, the invention provides an Rg1 supplement for use in rejuvenation of muscle cells in a subject, which comprises, or consists essentially of, or consists of, (1) Panax ginseng extract or Panax notoginseng extract or a combination thereof and (2) Rosa roxburghii extract at an amount to reverse the aging of muscle cells in the subject, wherein the Rg1 supplement is standardized to contain 30%-40% of a total saponin, 0.6% to 2.0% of Vitamin C of, and 2.0%-4.0% of polyphenols, and a ginsenoside Rg1 as one indicator component ranging from 5 mg to 50 mg for one serving; and wherein the Rg1 supplement is administered to said subject after an acute bout of squat exercise.

In addition, the invention provides a method for reversing the aging of muscle cells after excise in a subject, which comprises administering to said subject the Rg1 supplement after his/her completing of an acute bout of aerobic exercise, wherein the Rg1 supplement comprises (1) Panax ginseng extract or Panax notoginseng extract and (2) Rosa roxburghii extract, and is standardized to contain 30% to 40% of a total saponin, 0.6% to 2.0% of Vitamin C, and 2.0% to 4.0% of polyphenols, and a ginsenoside Rg1 as one indicator component ranging from 5 mg to 50 mg for one serving.

In addition, the invention provides a method for rejuvenation of muscle cells in a subject, which comprises administering to said subject the Rg1 supplement after his/her completing of an acute bout of aerobic exercise.

On the other hand, the invention provides a use of an Rg1 supplement for manufacturing an active agent or a medicament for rejuvenation of muscle cells in a subject, wherein the Rg1 supplement comprises, or consists essentially of, or consists of, (1) Panax ginseng extract or Panax notoginseng extract or a combination thereof and (2) Rosa roxburghii extract at an amount to reverse the aging of muscle cells, wherein the Rg1 supplement is standardized to contain 30%-40% of a total saponin, 0.6% to 2.0% of Vitamin C of, and 2.0%-4.0% of polyphenols, and a ginsenoside Rg1 as one indicator component ranging from 5 mg to 50 mg for one serving; and wherein the Rg1 supplement is administered to said subject after an acute bout of squat exercise.

The term “Rg1 supplement” or “Rg1” as used herein refers to a supplement enriched with a compound having the chemical name: (3β, 6α, 12β)-3,12-Dihydroxydammar-24-ene-6,20-diyl bis-β-D-glucopyranoside, which is a major component of the root and stem of ginseng plant. Rg1 is known as a ginseng-based immunostimulant, which enhances neutrophil emigration and CD4′ lymphocyte activity [Lee et al., 2004; Zou et al., 2013].

According to the invention, the Rg1 supplement may be formulated using any standard technology or commonly used methods known to those skilled in the art. The Rg1 supplement comprises, or essentially consists of, or consists of, (1) Panax ginseng extract or Panax notoginseng extract, and (2) Rosa roxburghii extract, at a ratio of 4:1˜1:4, for example 1:1, which is standardized to contain 30%-40% of total saponins, 0.6%-2.0% of Vitamin C, and 2.0%-4.0% of total polyphenols, and a ginsenoside Rg1 as one indicator component ranging from 5 mg to 50 mg (e.g., 5 mg) for one serving; one example of which is the product with the brand name of ActiGin® supplied by NuLiv Science USA, Inc.

According to the invention, the Panax ginseng extract, Panax notoginseng extract, or Rosa roxburghii extract may be obtained with water, ethanol or a combination thereof, which may be obtained by any commonly used method or standard method. In the invention, either Panax ginseng or Panax notoginseng can be used to provide a ginsenoside Rg1 as one indicator component, which may be obtained from the extraction of the roots of Panax ginseng or Panax notoginseng with water or/and ethanol. In one particular example, the extraction of Panax ginseng or Panax notoginseng comprises the steps of:
washing the raw materials;
pulverizing the materials through a size 20 mesh screen and repeating the extraction of the materials with water to obtain a water extract;
centrifuging and concentrating the water extract, and running it through an absorptive resin column; and
collecting the eluent, further washing, and concentrating the extract as obtained prior to storage.

In the invention, the Rosa roxburghii extract may be obtained by the extraction with water and/or ethanol by any commonly used method or standard method. In one particular example, the Rosa roxburghii extract was obtained by the process of the steps of:
washing the fresh fruits of Rosa roxburghii, and repeating the extraction of the fruits with water at a low temperature in an anaerobic condition to obtain a crude extract;
filtering the crude extract and discarding the solid matter to obtain a filtrate;
evaporating the filtrate at a low temperature and in an anaerobic condition; and
purifying, drying and grinding the paste to a specific mesh size to obtain the final extract.

The term “effective amount” as used herein refers to an amount of a drug or pharmaceutical agent which, as compared to a corresponding subject who has not received such amount, results in an effect in treatment or prevention of a disease, disorder, or side effect, or a decrease in the rate of advancement of a disease or disorder. The term also includes within its scope amounts effective to enhance normal physiological function.

The term “a pharmaceutically acceptable carrier” as used herein refers to a carrier, diluent, or excipient that is physiologically acceptable, in the sense of being compatible with the other ingredients of the formulation and not deleterious to the subject to be administered with the composition. Any carrier, diluent or excipient commonly known or used in the field may be used in the invention, depending to the requirements of the formulation.

As known in the prior art, stem cell aging, characterized by elevated p16^{INK4a} expression, decreases cell repopulating and self-renewal abilities, which results in elevated inflammation and slow recovery against stress. Accordingly, in the invention the biopsied muscles were analyzed at baseline and 24 h after squat exercise in 12 trained men (22±2 y), who were administered with Placebo (PLA) or the Rg1 supplement (5 mg) 1 hour before a squat exercise, using a double-blind counterbalanced crossover design. It is found in the method that perceived exertion at the end of resistance exercise session was significantly lowered after the administration of the Rg1 supplement. In addition, the exercise doubled endothelial progenitor cells (EPC) (p<0.001) and decreased p16^{INK4a} mRNA to 50% of baseline (d=0.865, p<0.05) in muscle tissues, despite p16^{INK4a+} cell and beta-galactosidase⁺ (β-Gal⁺) cell counts being unaltered. The Rg1 supplement further lowered p16^{INK4a} mRNA to 35% of baseline with greater effect size than the PLA level (d=1.302, p<0.01) and decreased myeloperoxidase (MPO) mRNA to 39% of baseline (p<0.05). A strong correlation between MPO and p16^{INK4a} expression in muscle tissues was observed (r=0.84, p<0.001). It can be concluded in the invention that EPC in skeletal muscle doubled 1 day after an acute bout of resistance exercise. The exercised effects in lowering EPC aging and tissue inflammation were enhanced by the Rg1 supplement, suggesting the involvement of immune stimulation on EPC rejuvenation.

The present invention will now be described more specifically with reference to the following examples, which are provided for the purpose of demonstration rather than limitation.

### EXAMPLES

### Example 1 Preparation of the Herbal Composition According to the Invention

The Rg1 supplement according to the invention may be obtained by combining the extract of Panax ginseng or Panax notoginseng, and Rosa roxburghii extract, both of which were extracted with water and/or ethanol.

To obtain the water extract of Panax ginseng, the raw materials of Panax ginseng roots were washed and pulverized through a size 20 mesh screen; the water extraction was then centrifuged, and concentrated. The solution as obtained run through an absorptive resin column and the eluent was collected, further washed, and concentrated prior to storage.

To obtain the water extract of Rosa roxburghii, the fruits of Rosa roxburghii were washed and repeatedly extracted with mater at low temperature in an anaerobic condition. Then, the solid matter was filtered and discarded to obtain a filtrate; and the filtrate was then subject to an evaporation at low temperature in an anaerobic condition to obtain a paste. The paste as obtained was purified, dried and ground to a specific mesh size to obtain the final extract.

The Panax ginseng extract and the Rosa roxburghii extract were mixed at the ratio of 4:1-1:4, and then standardized to contain 30%-40% of total saponins, 0.6%-2.0% of Vitamin C, and 2.0%-4.0% of total polyphenols, and a ginsenoside Rg1 as one indicator component ranging from 5 mg to 50 mg (e.g., 5 mg) for one serving, which was called as “the Rg1 supplement” or “Rg1” as used for the following human trials. In the examples, the Rg1 supplement was obtained from NuLiv Science USA, Inc. (CA, USA).

### Example 2

Participants

Twelve healthy young men 20-23 years of age (weight: 51-95 kg; height: 161-190 cm) with \dot{V}O2max ranging between 43-55 ml⁻¹ kg⁻¹ min⁻¹ volunteered to participate in this study. Participants were untrained and recreationally active non-smokers. They were fully informed of the risks and discomfort associated with the study, and all provided written consent before participation. This study was conducted in accordance with the guidelines contained in the Declaration of Helsinki and was approved by the Institutional Review Board of University of Taipei, Taipei, Taiwan.

Experimental Design

A placebo-controlled, counter-balanced, crossover study was conducted. Participants were randomized into one of two groups: PLA (5 mg of cellulose) and Rg1 (5 mg). Rg1 was obtained from NuLiv Science USA, Inc. (CA, USA). Participants randomly assigned to the PLA group received cellulose before trial one, and Rg1 before trial two. Accordingly, participants randomly assigned to the Rg1 group received Rg1 before trial one and cellulose before trial two. Trials one and two were separated by ten days. The Rg1 and cellulose were provided in capsules 1 h before exercise. Capsules were coded by number for later identification. Exercise consisted of 1 h of continuous cycling at 70% \dot{V}O2max on a cycle ergometer (Monark 839E, Stockholm, Sweden).

Experimental Protocol

All participants were familiarized with the experimental procedures and equipment before testing started. Participants completed a \dot{V}O₂max test using a graded exercise protocol on a cycle ergometer one week before starting the experimental trials. All participants consumed a standard isocaloric diet 12 h prior to each experimental trial to limit any potential dietary effect that could influence the outcome of the study. Participants orally ingested 5 mg of Rg1 or PLA 1 h before cycling. After exercise, participants consumed a high carbohydrate meal (1.5 g carbohydrates per kg body weight: carbohydrate 80%, fat 8% and protein 12%; glycemic index: 80) within 10 min at the start of a 3-h recovery period. Water was provided ad libitum during and after the meal.

Muscle Biopsy

Muscle samples were taken from vastus lateralis muscle before (Pre), immediately (0 h) and 3 h after the 1-h cycling exercise protocol. Biopsies were performed under local anesthesia (2% lidocaine) by a certified physician using an 18G Temno disposable cutting needle (Cardinal Health, Waukegan, Ill., USA). Biopsies were taken from the vastus lateralis 3 cm in depth and 20 cm proximal to the knee. The baseline muscle biopsy (Pre) was conducted 3-4 weeks before the start of the first 1 h cycling exercise test. Two consecutive muscle biopsies were performed immediately (0 h) after and 3 h after each cycling test. The 0 h and 3 h biopsies were taken from opposite legs, but at the same position on the vastus lateralis. A portion of each muscle sample was immediately frozen in liquid nitrogen and stored at −70° C. until analyzed. The rest of the sample was immediately placed in a conical vial containing 10% formalin and used for immunohistochemical analysis. Paraffin-embedded tissue was sectioned no later than 3 h following muscle sample collection.

Immunohistochemistry

Serial sections of paraffin-embedded tissue were cut and analyzed for distribution of p16^{INK4a} and CD34 in the vastus lateralis muscle. Paraffin sections (8 μm thick) were labeled using immunohistochemistry for binding of human monoclonal antibody p16INK4a (1:200, ab108349; Abcam, Cambridge, Mass., USA) and CD34 (1:200, ab81289; Abcam, Cambridge, Mass., USA). Immunofluorescence was used to detect regenerative macrophage CD163 (1:400, ab87099; Abcam, Cambridge, Mass., USA) infiltration in the vastus lateralis after exercise. Optical images were analyzed using ImageJ (NIH, Bethesda, Md.). Positive markers within cells were quantified and expressed as positive signal number/total skeletal muscle fiber number (%). An average of 600 muscle fibers per slide were included for analysis. All analyses were conducted by a specialist at the University of Taipei and a certified pathologist from Taipei Institute of Pathology with similar results. An additional 36 muscle biopsied samples were used to assess correlation between p16INK4a positive cells and β-galactosidase positive cells (1:150, NBP2-45731, Novus Biologicals, CO, USA).

Quantitative PCR

RNA was extracted from ˜15 mg of skeletal muscle using TRI Reagent (T9424-200) (Sigma, St. Louis, Mo., USA) for homogenization, followed by isopropanol precipitation, two ethanol washes, drying, and suspension in 20 μl nuclease-free water. One microgram of RNA in a total volume of 20 μl was reverse transcribed to cDNA using iScript cDNA Synthesis Kit (#170-8890) (Bio-Rad, Hercules, Calif., USA). Real-time PCR was performed using MyiQ Single Color Real-Time PCR Detection System (Bio-Rad, Hercules, Calif., USA), TaqMan Probe (Sigma-Aldrich, Singapore) and iQ Supermix kit (#170-8860) (Bio-Rad, Hercules, Calif., USA). The PCR conditions for all genes consisted of one denaturing cycle at 90° C. for 30 s, annealing at 60° C. for 60 s and elongation at 72° C. for 60 s. At the end of the PCR the samples were subjected to a melting curve analysis. To control for any variations due to efficiencies of the reverse transcription and PCR, 18S ribosomal RNA was used as an internal standard to determine relative expression levels of the target mRNAs. The primers and probes used to amplify target mRNA are

18S ribosomal (18S):

Forward (5′-3′): ACAGGATTGACAGATTGAT AGCTC,
Reverse (5′-3′): TCGCTCCACCAACTAAGA ACG,
Probe (5′-3′): TGCACCACCACCCACGGAATC GAG;
IL-10:
Forward (5′-3′): CTTCCCTGTGAAAAC AAG,
Reverse (5′-3′): AGACCTCTAATTTATGTCC TA,
Probe (5′-3′): AGTCGCCACCCTGATGTCTC;
VEGF:
Forward (5′-3′): TGAGATCGAGTACATCTTC AAGCC,
Reverse (5′-3′): GGCCTTGGTGAGGTTTGA TCC,
Probe (5′-3′): CCTGTGTGCCCCTGATGCGAT GCG;
PGC-1α:
Forward (5′-3′): CGAGGAATATCA GCACGAGAGG,
Reverse (5′-3′): CATAAATCACAC GGCGCTCTTC,
Probe (5′-3′): TGCCTTCTGCCTCTG CCTCTCCCTC.

A Sequence Listing entitled “METHOD AND COMPOSITION FOR REJUVENATION OF MUSCLE CELLS” created on or about Aug. 19, 2022 (3 KB long), of the primers and probes set forth hereinabove is attached hereto in ASCII text format. The above-noted Sequence Listing is being incorporated into this specification by reference in its entirety.

Serum LDH, Myoglobin and TBARS

A colorimetric assay kit was used to detect serum LDH activity according to the manufacturer's instructions (BioVision, #k726-500, CA, USA). Myoglobin was measured by ELISA using a commercially available kit (Immunology Consultants Laboratory, E-80MY, OR, USA). Serum samples were also used after further dilution for measurement of TBARS using a commercially available kit (Cayman Chemical, No. 10009055, MI, USA).

Statistical Analysis

All data are expressed as means±standard error. The data were analyzed using a two-factor repeated-measures ANOVA (SPSS 20.0). Post hoc paired comparison analysis was performed with the Fisher LSD method. Type I error of P≤0.05 was considered significant. P≤0.1 was considered moderately significant.

Results

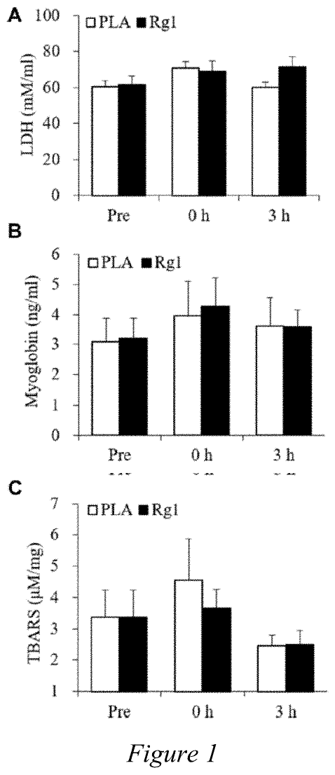

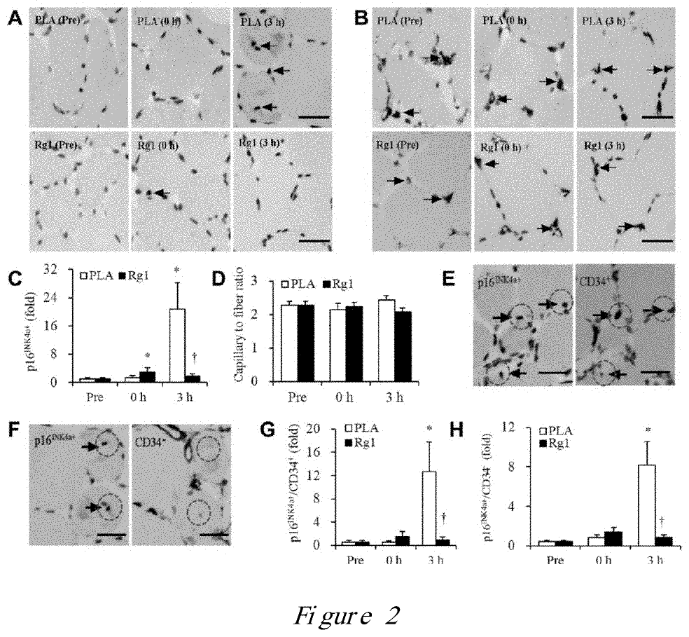

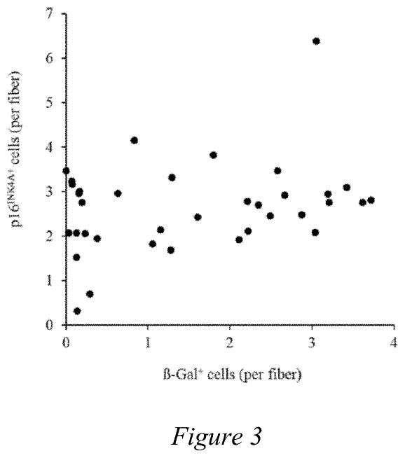

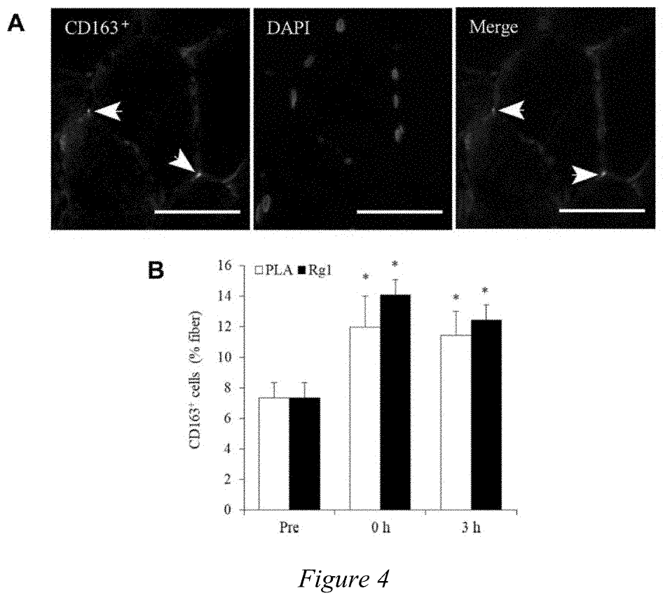

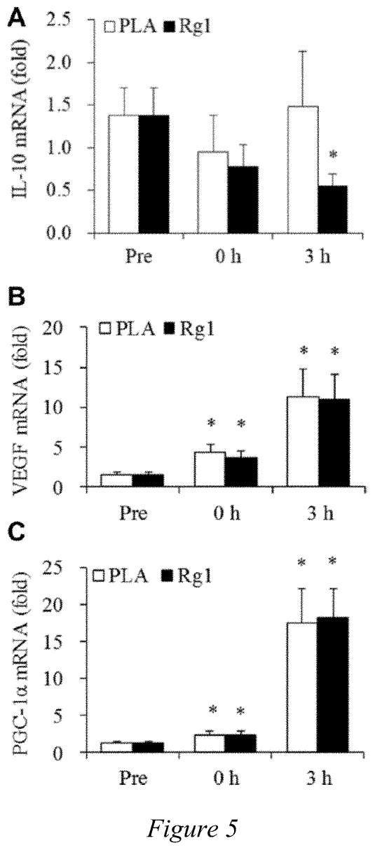

Endurance cycling at 70% \dot{V} O₂max for 1 h does not produce significant increases in circulating LDH and myoglobin (FIG. 1). The lipid peroxidation marker TBARS levels tends to increase by 35% during the PLA trial (FIG. 1C). No detectable change in p16^{INK4a+} senescent cells was observed immediately after exercise (PLA trial). However, ˜21-fold increases (P<0.01) in p16^{INK4a+} senescent cells of skeletal muscle occurred 3 h after exercise. During the Rg1 trial, ˜3-fold increases (P<0.05) in p16^{INK4a+} senescent cells of skeletal muscle were observed immediately exercise followed by ˜40% decline 3 h after exercise (P<0.05) (FIG. 2A, 2C). CD34⁺ cell-to-fiber ratio of skeletal muscle was not altered after exercise (FIG. 2B, 2D). A great portion of senescent cell accumulation in exercised muscle is contributed by increased senescent endothelial progenitor cells (p16^{INK4a+}/CD34⁺) (FIG. 2E, 2G). Approximately 40% of the increases is associated with other types of nucleated cells (p16^{INK4a+}/CD34⁻) localized mostly in necrotic myofibers (FIG. 2F, 2H). Dramatic increases in p16^{INK4a+} cells 3 h after exercise is potently lowered by Rg1 supplementation (P<0.05). A moderate correlation (r=0.29, p=0.08) was found between p16^{INK4a} positive cells and β-galactosidase positive cells of 36 biopsied muscle samples in men aged 20-26 y (FIG. 3). Aerobic cycling (70% \dot{V} O2max) increased CD163⁺ macrophage infiltration into human skeletal muscle (FIG. 4). Placebo (PLA) and Rg1 trials show a similar magnitude of cell infiltration (PLA: 0 h, +63%, P<0.05; 3 h, +56%, P<0.05; Rg1: 0 h, +92%, P<0.01; 3 h, +70%, P<0.01). Data for exercise response in IL-10 mRNA, VEGF mRNA and PGC-1α mRNA of skeletal muscle are shown in FIG. 5. IL-10 mRNA did not change after an acute bout aerobic exercise, while Rg1 significantly decreased IL-10 mRNA expression 3 h after exercise (−60%, P<0.05) (FIG. 5A). Both VEGF mRNA (FIG. 5B) and PGC-1α mRNA (FIG. 5C) increased significantly in challenged skeletal muscle. Similar increases were observed in VEGF mRNA of skeletal muscle for both PLA (0 h, +2-fold, P<0.01; 3 h, +7-fold, P<0.01) and Rg1 (0 h, +1-fold, P<0.05; 3 h, +7-fold, P<0.01) trials following exercise. PGC-1α mRNA also shows similar increases for PLA (0 h, +1-fold, P<0.05; 3 h, +13-fold, P<0.01) and Rg1 (0 h, +1-fold, P<0.01; 3 h, +14-fold, P<0.01) trials.

Discussion

Aerobic exercise induces VEGF expression and stimulates angiogenesis [Tsai et al., 2016]. During this process, endothelial progenitor cells are capable of selfreplicating to increase cell number to renew damaged endothelial cells in the capillaries [Urbich & Dimmeler, 2004]. However, oxidative DNA damage is increased after an acute bout of aerobic exercise [8, 16]. In this study, we found a considerable rise in the tumor suppressor p16^{INK4a} protein expression in endothelial progenitor cells of human skeletal muscle after an acute bout of aerobic exercise (70% V 02max). Furthermore, the supplement Rg1 accelerated the resolution of this stress response, evidenced by an earlier rise and fall of p16^{INK4a+} cell number in exercised muscle. Stress that causes DNA damage results in the increased expression of the p16^{INK4a} protein, which is responsible for inhibition of cell division [Sharpless, 2004]. Thus, the transient increases in p16^{INK4a+} protein expression may mirror the magnitude of stress-related DNA damage [Wu et al., 2019] and suggests a protective mechanism for maintaining genetic stability of replicable cells against aerobic exercise.

Increasing p16^{INK4a} protein expression is also known to stimulate tissue repair during inflammation [Sarkar-Agrawal et al., 2004; Serrano, 2014]. Inflammation is essential for recognition and phagocytic clearance of unhealthy cells that develops a senescence phenotype [Kay, 1975; Prata 2018; Sagiv et al., 2013] followed by a protracted period of cell regeneration [Tidball, 2017]. A recent study has reported an enhanced regenerative process after increasing p16^{INK4a+} mesenchymal stem cells during muscle inflammation [Chikenji et al., 2019]. Taken together, these recent findings suggest a new role of stress-induced p16^{INK4a} protein expression in inflammation-mediated muscle regeneration. Rg1 is an immunostimulant that activates macrophage [Fan et al., 1995] and advances the progression of inflammation from M1 to M2 phenotype in exercised human skeletal muscle [Hou et al., 2015]. Therefore, pre-exercise Rg1 intake is likely to potentiate the inflammation response (preconditioning) and result in an early resolution of the exercise stress response. M2 macrophage (CD163+) polarization occurs during the later period of inflammation, which is responsible for cell regeneration of muscle tissue after physical challenge [Tidball, 2017; Wang et al., 2015]. However, massive increases in p16^{INK4a+} endothelial progenitor cells observed in the study was not directly associated with an exercise induced increment of M2 macrophage in skeletal muscle. The magnitude of increase in M2 macrophage was similar for PLA and Rg1 trials, while the exercise induced response of p16^{INK4a+} cells in human skeletal muscle was lower than that in the PLA trial. However, our results do not preclude the possibility that accumulation of p16^{INK4a+} endothelial progenitor cells directly increase M2 macrophage activity in human skeletal muscle. A limitation of the study is the difficulty in determining whether increased p16^{INK4a+} cells 3 h post-exercise was completely attributed cell senescence or simply due to a reversible induction of p16^{INK4a} protein expression of well-functioned endothelial progenitor cells. Cellular senescence is featured by an irreversible form of cellcycle arrest after prolonged stress. It is not possible to determine whether p16^{INK4a+} cell number is representing of irreversible cell-cycle arrest in human muscle tissue, since both p16^{INK4a+} cell accumulation and p16^{INK4a+} cell removal (immune clearance) can occur in the same muscle tissue following exercise. In this study, a moderate correlation between p16^{INK4a+} cells and β-galactosidase positive cells in 36 muscle samples suggests that p16^{INK4a} is not a perfect tissue senescence marker. This is in agreement with a previous study [Dungan et al., 2020]. Whether p16^{INK4a} is a reliable cell senescence marker in human tissues cannot be settled in this study. Another limitation is inadequate time points for muscle biopsies, which prevented us from delineating the timings of the rise-and-fall pattern for p16^{INK4a+} cell accumulation in exercising muscle. The acute response of p16^{INK4a} protein expression in endothelial cells of skeletal muscle after aerobic exercise is in sharp contrast to what has been observed after resistance exercise. We and others have previously shown decreased p16^{INK4a+} cells in muscle tissue of untrained active women and men after resistance training [Yang et al., 2018; Justice et al., 2018]. Time required for resolution of the stress response and inflammation during and after exercise is associated with the magnitude of tissue damage, and varying by mode, intensity and duration of exercise. Furthermore, the local tissue response is influenced by distribution of the whole-body immune resource (white blood cells and stem cells from bone marrow). For example, aerobic exercise exerts a major challenge to the cardiopulmonary system in addition to skeletal muscle. The majority (>60%) of white blood cells and stem cells are harbored in the lungs for constant local repair and regeneration [Adams et al., 2011; Rochefort et al., 2005]. Aerobic exercise creates a competition between the lungs and muscle for immune resources for cell turnover [Adams et al., 2011]. In contrast, resistance exercise generates little challenge to the lungs, yet eccentric muscle contractions induce a substantial amount of muscle damage, which attracts more immune cells compared with aerobic exercise. Therefore, infiltration of immune cells into skeletal muscle after resistance exercise would be less likely to be compromised by competition with the lungs as occurs with aerobic exercise. Such differences can produce distinct rise-and-fall patterns for p16INK4a+ cells in human skeletal muscle [Prata et al., 2018; Chang et al., 2016], and possibly explain the delayed response of p16INK4a+ cells in human skeletal muscle after aerobic exercise.

### Conclusions

In the examples, a considerable increase was observed in p16INK4a protein expression of endothelial progenitor cells in human skeletal muscle 3 h after aerobic exercise at 70% V 02max. The result suggests that the increased p16INK4a expression is a protective mechanism to maintain genetic stability of replicable cells during regenerative phase after aerobic stress. Early resolution of this stress response occurs when the supplement Rg1 is orally taken 1 h before exercise.

### Example 3

Materials and Methods

Ethical Approval

The study was approved by the Institutional Review Board of University of Taipei, Taipei, Taiwan (IRB-2017-041). All experimental procedures were conducted in accordance with the Declaration of Helsinki. Participants were given full explanation of the purpose, experimental procedure, and the potential risks of participation. Written informed consent was received prior to the commencement of the study.

Study Design

To assess the muscle response after an acute bout of squat exercise under PLA- and Rg1-supplemented conditions, a randomized, double-blind placebo-controlled, counter-balanced crossover study design was conducted with a 3-week washout period. All participants consumed a standardized balanced nutrition shake (Ensure, Enlive, Abbott Nutrition, Chicago, Ill., USA) 12 h before the acute exercise challenge. Following the 12-h overnight fast, participants were randomized to the PLA- and Rg1-trials in a counter-balanced order and the PLA- and Rg1-supplements were orally delivered one hour before squat exercise. Capsules containing PLA (cornstarch) or Rg1 were orally delivered to participants via a drink (Herbalife Formula One shake, California, USA) one hour before the squat exercise in the morning. Participants could not distinguish the difference of PLA- and Rg1-supplements during oral delivery. A Rg1 dosage of 5 mg was used in this study (NuLiv Science USA, Inc. in California, USA), based on a previous report [Wu et al., 2019].

Muscle biopsies were performed at baseline and 24 h after squat exercise. Rating of Perceived Exertion (RPE) was self-reported immediately after the exercise [Helms et al., 2016]. Each participant completed the 1 repetition maximum (1-RM) assessment, as a maximum muscle strength assessment, three weeks prior to squat exercise. The baseline muscle biopsy was performed >10 days before squat exercise. Participants were informed to stop any form of their own training activity for a week before and after the study. Post-exercise muscle biopsy was performed 24 h after the squat exercise (25 h after PLA or Rg1 supplementation) to allow a short recovery.

Maximum Leg Strength

Maximum muscle strength was determined as 1-RM according to previous study [Helms et al., 2016]. After a warm-up exercise consisted of dynamic stretching exercises, a specific warm-up included a set of 10 repetitions with 50% of an estimated 1-RM (according to perceived capacity), a set of 5 repetitions with 75% of the estimated 1-RM, and a final set of 1 repetition with 90-95% of the estimated 1-RM. After a 5-min rest period, participants completed 3 to 5 attempts with progressively heavier weights (˜5%), interspersed with 3-5 min rest intervals, until a 1-RM was achieved. Participants were instructed to adopt a shoulder width stance in keeping with their normal squat stance, descend in a controlled manner, avoid bouncing at the bottom position, maintain as near a vertical torso as possible, and feet always flat on the ground.

Squat Exercise

A trainer instructed the exercise protocol for participants in a weight training room to ensure the consistency of the challenge. Each participant was required to perform a back-squat exercise using a barbell and a power half squat rack. Participants were advised to lower the barbell until their knees reach 90°, by having the hamstrings touching a resistance band placed in the power half squat rank. Each participant completed his own set of warm up followed by a structured set of 8 repetitions (50% 1-RM) of barbell back squat. The squat exercise consisted of 6 sets of 8 repetitions (70% 1-RM) with 90 s rest interval between sets.

Self-Perceived Measurements

RPE was self-reported immediately after the completion of the exercise. Participants reported the RPE by observing a numerical scale, ranging from 1 “rest” to 10 “maximal effort”. The RPE questionnaire was also based on repetitions in reserve (RIR) for resistance exercise [Day et al., 2004].

Before (Pre) and after (Post, 24 h, 48 h, 72 h) the exercise protocol, perceived muscle soreness/pain were assessed with the visual analog scale (VAS), using a continuous 10-cm scale anchored by two verbal descriptors labeled from the left (no pain) to the right (worst possible pain) [Sriwatanakul et al., 1982].

Muscle Biopsy

The muscle biopsy procedure was conducted by an experienced physician using a 14-gauge Temno disposable cutting needle (REFT149, CareFusion, Vernon Hills, Ill. U.S.A) inserted into the vastus lateralis at 3 cm depth and ˜20 cm proximal to kneecap. Two biopsied muscle samples were collected at each time point. Local anesthesia (2% lidocaine hydrochloride) was administered prior to the procedure. The baseline biopsy was collected from the right leg, more than 10 days before the exercise challenge. After collection, muscle tissue was immediately placed into 20 ml glass vial containing 10% formalin and then embedded into paraffin wax block. This formalin-fixed paraffin wax-embedded (FFPE) tissues were then cut using serial sectioning protocol and mount on glass slides for staining.

Immunohistochemistry (IHC) Staining

IHC analysis as a semi-quantitative methods was conducted by a pathologist at the Taipei Institute of Pathology (Taipei, Taiwan). The XT UltraView DAB v3 and BenchMark XT IHC/ISH Staining Module protocol (Ventana Medical Systems, AR, USA) were used to detect expression of monoclonal antibodies p16_{INK4A} (1:200, ab108349; Cambridge, Mass., USA), β-Gal (1:150, NBP2-45731; Novus Biologicals, USA), CD34 (Ventana Medical Systems, USA), and CD4 (1:100, ab133616; Cambridge, Mass., USA) in muscle serial sections. The binding of primary antibody to a specific antigen was located by enzyme labelled secondary antibodies. The complex was then visualized with hydrogen peroxide substrate and 3, 3′-diaminobenzidinetetrahydrochloride (DAB) chromogen, which produced brown precipitates. Pale to dark blue coloration represents cell nuclei, whereas brown coloration represents positively stained antigens.

For p16_{INK4a} detection, slides were deparaffinized, washed twice for 5 min in TBS plus 0.025% Triton X-100 and blocked in 10% normal serum with 1% BSA in TBS for 2 h at room temperature. Slides were drained before applying p16_{INK4a} antibody diluted in TBS with 1% BSA and incubated overnight at 4° C. After two 5-min rinses with TBS 0.025% Triton, the slides were incubated in 0.3% H₂O₂ in TBS for 15 min. Enzyme-conjugated anti-rabbit secondary antibody was applied to the slide, diluted in TBS with 1% BSA, and incubated for 1 h at room temperature. After a 5-min rinse in tap water, the slides were counterstained with hematoxylin.

For β-Gal detection, muscle paraffin sections (2 μm thick) were deparaffinized and rehydrated with xylene and ethanol (100%, 95%, 70% 50% and deionized water). Slides were boiled in 10 mM sodium citrate buffer (pH 6.0), cooled on bench top (30 min), and immersed in distilled water (5 min). Tissue sections were quenched with 3.0% hydrogen peroxidase in methanol for 15 min, washed in distilled water (5 min), washed twice with permeabilization buffer containing 1% animal serum and 0.4% Triton X-100 in Phosphate-buffered saline (PBS-T), and incubated with 5% animal serum in PBS-T for 30 min at room temperature. Primary antibody (diluted in 1% animal serum in PBS) was added, incubated at room temperature for 1-2 h, and overnight incubation at 4° C. in a humidified chamber. Sections were washed twice with 1% serum in PBS-T for 10 min before adding anti-mouse secondary antibody to each section. The sections were incubated at room temperature for 1 h before washing twice with 1% serum PBS-T for 10 min each. DAB working solution was prepared and applied to tissue that causes chromogenic reaction. Sections developed brown color with positive reaction. Slides were then immersed in deionized water twice for 2 min then were counterstained with hematoxylin. Pale to dark blue coloration represents cell nuclei, whereas brown coloration represents positively stained antigens.

CD4 detection used similar antigen retrieval method as p16_{INK4a}. The samples on the slides were then incubated with the monoclonal anti-CD4 antibody at room temperature for 15 min. The samples were washed and then incubated with the anti-rabbit secondary antibody for 10 min. After color development with 3,3′-diaminobenzidine at room temperature for 10 min, the sections were counterstained with hematoxylin for 15 min, dehydrated, and mounted according to the standard protocol.

Image Analysis

The muscle cross-sectional area on the slides were reviewed and captured at 10× magnification (BX50 Olympus microscope, Tokyo, Japan) using MShot Image Analysis System v1.0. The analyses were conducted using manual inspection (Image J, National Institute of Health, USA) of 6 visual fields that contains the most compact and complete muscle fibers (>50). Positively stained markers were quantified via cells expressing brown coloration as antigen. Positive marker criteria include: (a) muscle cell must be whole and intact; (b) the positive marker must have brown (antigen) stained coloration; (c) the positive marker must be intact/attached with muscle cell; (d) colocalization of positive marker must meet criteria for both markers fulfilling (a), (b), and (c) concurrently. Total positive signals (p16_{INK4A+}, CD34+, β-Gal+, CD4+) and colocalization number of positive signals of p16_{INK4A+}/CD34+, p16_{INK4A+}/CD4+ of the images were measured. Positive cell counts were normalized to fibers number.

Quantitative PCR

RNA was extracted from ˜15 mg of biopsied muscle using the RNeasy kit (QIAGEN 74104) after a 60-s homogenization in QIAzol Lysis Reagent (QIAGEN, Hilden, Germany, 79306). One microgram of RNA in a total volume of 20 μl was reverse-transcribed to cDNA using iScript cDNA Synthesis Kit (#170-8890) (Bio-Rad, Hercules, Calif., USA). Real-time PCR was performed using CFX Connect Real-Time PCR Detection System (Bio-Rad, Hercules, Calif., USA), PrimePCR™ Probe Assay (Bio-Rad, Hercules, Calif., USA) and iQ Supermix kit (#170-8862) (Bio-Rad, Hercules, Calif., USA). The cycling parameters were: 95° C. for 3 min, then 50 cycles at 95° C. for 10 s and 58° C. for 30 s. Gene expression, normalized to the geometric mean of a housekeeping genes (RPP30), was quantified using the 2-(ΔCt) method and expressed as fold difference relative to the RPP30. The primers and probes used to amplify target were are supplied from Bio-Rad PrimePCR™ Probe Assay: p16_{INK4a} (or CDKN2A) (Assay ID: qHsaCEP0057827), CD34 (Assay ID: qHsaCIP0026476), GLB1 (Assay ID: qHsaCEP0057625), MPO (Assay ID: qHsaCEP0049167), and RPP30 (Assay ID: qHsaCEP0052683).

Statistical Analysis

All results were presented as mean±standard error (SE). Type 1 error equal or less than 5% for comparing mean difference was considered significant. Two-way ANOVA with repeated measure was used to determine the main effect and interactive effects of time (between-factor) and supplement (within-factor). The % change after exercise from baseline between trials were analyzed using paired t-test. Effect size were measured using Cohen's d. The d values above 0.5 and 0.8 are considered medium and large effect, respectively. Pearson's correlation was used to determine the magnitude of association between variables.

Results

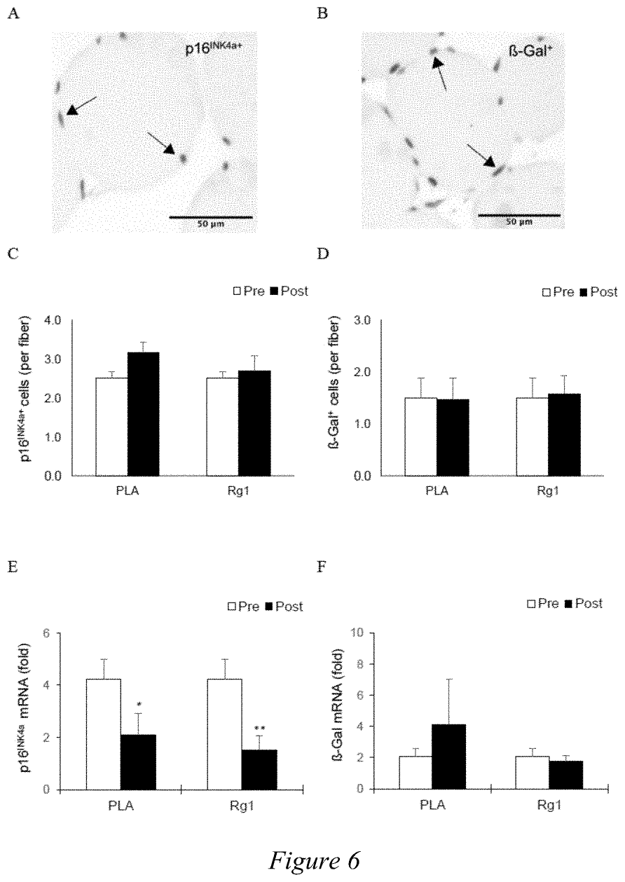

IHC data (expressed as positive cell number per myofiber) for p16_{INK4a+} cells and β-Gal+ cells of muscle cross-sections are shown in FIG. 6. The p16_{INK4a+} cells and β-Gal+ cells were located surrounding myofiber (FIGS. 6A and 6B) and were unchanged 24 h following resistance exercise under both PLA- and Rg1-supplemented conditions (FIGS. 6C and 6D). Squat exercise decreased p16_{INK4a} mRNA of vastus lateralis muscle to 49% (d=0.865, p=0.034) of baseline in the PLA-trial and further decreased to 35% (d=1.302, p=0.007) (FIG. 6E) of baseline in the Rg1-trials, respectively (main effect of exercise, p=0.002). Exercise effect on β-Gal mRNA was not significant for both PLA- and Rg1-trials.

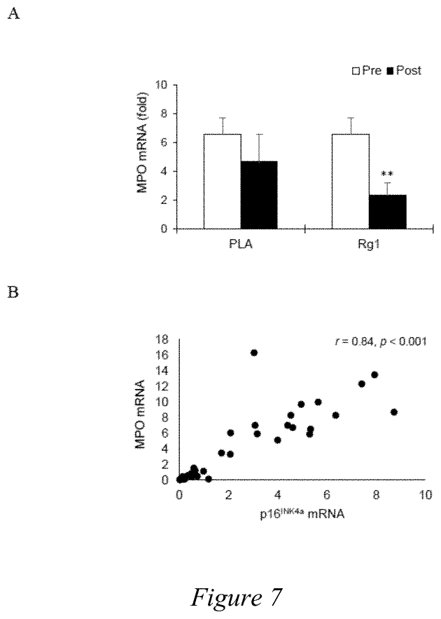

MPO mRNA (neutrophil marker) also decreased 24 h after squat exercise (main effect, p=0.026). Pre-exercise Rg1 supplementation induced greater decreases in MPO mRNA than PLA trial (PLA: d=0.382, p=0.196; Rg1: d=1.330, p<0.01). When all muscle tissues were pooled together, a high correlation was found between MPO mRNA and p16_{INK4a} mRNA (r=0.84, p<0.001). (FIG. 7B). Lower correlation was found between MPO mRNA and β-Gal mRNA (r=0.56, p=0.001). Rg1 decreased subjective perceived effort during resistance exercise without changes in muscle soreness. RPE after squat exercise was significantly lower (d=1.195, p=0.007) for the Rg1-supplemented trial (6±0.6 AU) than the PLA-supplemented trial (8±0.3 AU). However, no significant differences were found in the post-exercise muscle soreness (VAS for pain) between both trials (d=0.065, p=0.881).

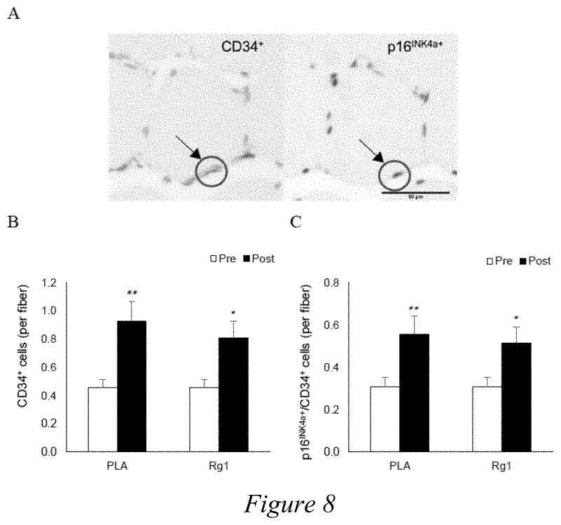

Some of p16^{INK4a+} cells in the muscle cross-section are colocalized with EPC (CD34+ cell) (FIG. 8A). EPC in muscle tissue almost doubles 24 h after resistance exercise for both PLA- and Rg1-supplemented trials (PLA: d=1.383, p<0.001; Rg1: d=1.095, p=0.015) (FIG. 8B). Despite unchanged total p16_{INK4a+} cells (positive cell per fiber) in muscle after exercise, p16^{INK4a+}/CD34+ cells were selectively elevated during both PLA and Rg1 trials (PLA: d=1.047, p=0.003; Rg1: d=0.933, p=0.010) (FIG. 8C).

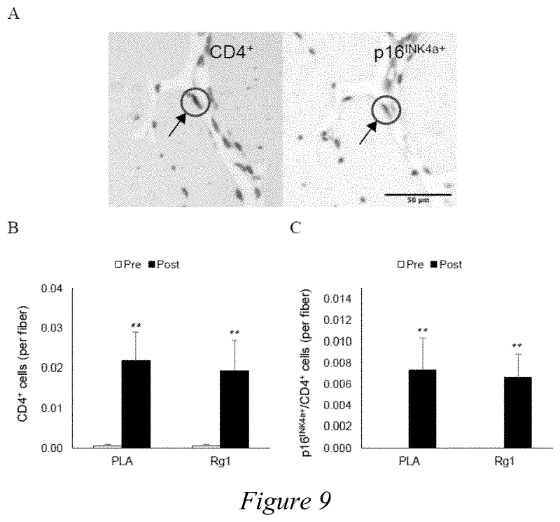

Some of p16_{INK4a+} signals in the muscle cross-section are colocalized with CD4+T lymphocytes (FIG. 9A). At baseline, CD4+T lymphocytes were rarely detectable (0.0005-0.02 positive signal per fiber) in unchallenged muscle and increased abruptly 24 h after exercise for both PLA and Rg1 trials (PLA: d=1.383, p=0.007; Rg1: d=1.095, p=0.002) (FIG. 9B). Similarly, p16_{INK4a+}/CD4+ T cells was completely absent at baseline and increased 24 h after the exercise (FIG. 9C).

Discussion

The key finding of the study is a doubled EPC and decreased p16^{INK4a} mRNA in human skeletal muscle 1 day after squat exercise. No change in p16_{INK4a+} cell number is probably associated with low levels of p16^{INK4a+} senescent cell number in muscle tissues of men with training experience [Justice et al., 2018; Yang et al., 2018]. It is likely that all replicable cells in the muscle tissue express a wide spectrum of p16^{INK4a} mRNA levels. Therefore, p16^{INK4a} mRNA seems to be a more sensitive biomarker to detect the level of tissue senescence in human skeletal muscle [Sharpless, 2004], compared with the semi-quantitative IHC analysis of p16^{INK4a+} cell number [Dungan et al., 2020]. The Rg1 supplementation before exercise results in greater effect size in the reductions of MPO mRNA and p16^{INK4a} mRNA in the exercised muscle, suggesting an involvement of immune stimulation in lowering cellular senescence after exercise.

Another novel finding of the study is a strong correlation (r=0.84) between p16_{INK4a} and MPO expression in human muscle tissues. This result supports the notion that cellular senescence attracts neutrophil infiltration to elevate muscle inflammation, suggested by animal and in vitro studies [Lasry & Ben-Neriah, 2015; Chikenji et al., 2019]. MPO is expressed specifically in neutrophils [Amanzada et al., 2011] which infiltrates in muscle tissue during phagocytic phase of inflammation [Tidball, 2017]. Low MPO expression found in this study may mirror a reduced demand for neutrophil-mediated phagocytosis after sufficient period of tissue renewal in human muscles [Tidball, 2017]. This finding implicates an immunity sparing effect of exercise by rejuvenating muscle tissues. Among the same men, lower correlation was observed between β-Gal and MPO mRNA, which suggests that β-Gal expression may not be a sensitive biomarker to reflect the magnitude of cellular senescence of human skeletal muscle.

Exercise induces a brief surge of EPC in blood in 6 h and quickly diminished to pre-exercise baseline within 24 h [Ribeiro, 2017]. In this study, we provide a novel evidence of an increased EPC in human skeletal muscle 24 h after resistance exercise. This local tissue adaptation suggests that bone marrow-derived EPC were seeded and proliferated in exercised skeletal muscle after their release into circulation. EPC contributes to fast regeneration of vascular endothelial cells and donates nucleus during myofiber regeneration [Tamaki et al., 2002]. Therefore, increasing EPC availability in tissues provides adaptive advantage in accelerating repair process for fast replacement of short-lived endothelial cells against daily challenges [Vasa et al., 2001]. This finding of EPC expansion in exercised human skeletal muscle provides a novel mechanism which explains the benefit of resistance exercise on vascular health and skeletal muscle adaptability.

CD4⁺ lymphocytes are immune cells that target mutated cells [Goswami & Awasthi, 2020] which are inherently produced during cell proliferation at high quantity in inflamed tissues. In the present study, CD4+ lymphocytes were rarely detectable in participants' muscles at baseline but expanded abruptly 24 h after an acute bout of squat exercise (FIG. 9). Massive expansion of CD4+T lymphocytes had also been reported as a feature of supercentenarians against tumors and viruses [Hashimoto et al., 2019]. The increased CD4+ cells are presumably protective to remove randomly mutated cells during high rate of cell turnover in exercised skeletal muscle. The cytotoxic effect of human CD4+ lymphocyte has been widely reported [Goswami & Awasthi, 2020], which implicates its function for senescent cell turnover to alter age profile of stem cell population in challenged tissues. Despite no significant difference in CD4⁺ lymphocyte number between PLA- and Rg1-supplemented conditions, greater magnitudes of senescence-reducing effect and decreased MPO mRNA in the exercised muscle after the Rg1 supplementation (FIG. 6) may be associated with activation of cytotoxic function (not number) of CD4′ lymphocytes and phagocytosis by neutrophils.

The cell cycle inhibitor p16_{INK4a} impedes cell division and plays an important role in decreasing incidence of DNA mutation [Sharpless, 2004]. Selective upregulation of p16_{INK4a} in proliferating cells after an acute bout of resistance training highlights a negative feedback mechanism to prevent uncontrollable cell regeneration triggered by exercise. This response may be important to maintain genetic fidelity during training adaptation. In addition, senescence has been thought as an essential stimulus to promote growth and development of young tissue [Storer, 2013]. The major limitation of the study is insufficient biopsy time point to delineate the dynamical change of p16_{INK4a} expression, which restricts our physiological interpretation of its expression after an acute bout of exercise. Muscle damage is occurred followed by a period of cell renewal. Therefore, a rise and fall pattern of such response may cause opposing outcome when aging and death of challenged were occurred at earlier time point during and after exercise challenge. We could not preclude a possibility that cellular senescence is acutely increased during and immediately post exercise to induce inflammation mechanism for enhancing phagocytosis and cell regeneration to rejuvenate the tissue [Chikenji et al., 2019].

### Conclusion

The current study found an EPC expansion together with decreased cellular senescence level in human skeletal muscle 24 h after an acute bout of squat exercise. The decreased p16_{INK4a} mRNA level occurs in absence of changes in p16_{INK4a+} cell number of muscle tissue in men. A strong correlation between p16_{INK4a} and MPO expression provides a support for a close link between cellular senescence and inflammation in human tissues. Pre-exercise Rg1 supplementation decreased MPO mRNA and exerts a greater effect size in senescence-lowering effect to muscle tissues suggesting the role of immune stimulation in stem cell rejuvenation induced by exercise.

While this specification contains many specifics, these should not be construed as limitations on the scope of the invention or of what may be claimed, but rather as descriptions of features specific to particular embodiments or examples of the invention. Certain features that are described in this specification in the context of separate embodiments or examples can also be implemented in combination in a single embodiment.

### REFERENCES

1. Adams G R, Zaldivar F P, Nance D M, Kodesh E, Radom-Aizik S, Cooper D M (2011). Exercise and leukocyte interchange among central circulation, lung, spleen, and muscle. Brain Behav Immun. 2011; 25:658-66. doi.org/10.1016/j.bbi.2011.01.002
2. Aicher, A., Adler, K., Urbich, C., Martin, H., Dimmeler, S. (2001). Number and migratory activity of circulating endothelial progenitor cells inversely correlate with risk factors for coronary artery disease. Circ Res, 89(1), E1-7. doi:10.1161/hh1301.093953
3. Amanzada, A., Malik, I. A., Nischwitz, M., Sultan, S., Naz, N., & Ramadori, G. (2011). Myeloperoxidase and elastase are only expressed by neutrophils in normal and in inflamed liver. Histochem Cell Biol, 135(3), 305-315. doi:10.1007/s00418-011-0787-1
4. Ashcraft K A, Peace R M, Betof A S, Dewhirst M W, Jones L W (2016). Efficacy and mechanisms of aerobic exercise on cancer initiation, progression, and metastasis: a critical systematic review of in vivo preclinical data. Cancer Res. 2016; 76:4032-50. doi.org/10.1158/0008-5472.CAN-16-0887 PMID:27381680
5. Boquoi, A., Arora, S., Chen, T., Litwin, S., Koh, J., & Enders, G. H. (2015). Reversible cell cycle inhibition and premature aging features imposed by conditional expression of p16Ink4a. Aging Cell, 14(1), 139-147. doi:10.1111/ace1.12279
6. Burton D G, Stolzing A. (2018). Cellular senescence: immunosurveillance and future immunotherapy. Ageing Res Rev. 2018; 43:17-25. doi.org/10.1016/j.arr.2018.02.001 PMID:29427795
7. Campisi, J. (2005). Senescent cells, tumor suppression, and organismal aging: good citizens, bad neighbors. Cell, 120(4), 513-522. doi:10.1016/j.cell.2005.02.003
8. Chang J, Wang Y, Shao L, Laberge R M, Demaria M, Campisi J, Janakiraman K, Sharpless N E, Ding S, Feng W, Luo Y, Wang X, Aykin-Burns N (2016). Clearance of senescent cells by ABT263 rejuvenates aged hematopoietic stem cells in mice. Nat Med. 2016; 22:78-83. doi.org/10.1038/nm.4010 PMID:26657143
9. Chikenji, T. S., Saito, Y., Konari, N., Nakano, M., Mizue, Y., Otani, M., & Fujimiya, M. (2019). p16(INK4A)-expressing mesenchymal stromal cells restore the senescence-clearance-regeneration sequence that is impaired in chronic muscle inflammation. EBioMedicine, 44, 86-97. doi:10.1016/j.ebiom.2019.05.012
10. Coussens, L. M., & Werb, Z. (2002). Inflammation and cancer. Nature, 420(6917), 860-867.doi:10.1038/nature01322
11. Crisan, D., Topalovski, M., & O'Malley, B. (1996). Myeloperoxidase mRNA analysis in acute lymphoblastic leukemia. Diagn Mol Pathol, 5(4), 236-248. doi:10.1097/00019606-199612000-00003
12. Day, M. L., McGuigan, M. R., Brice, G., & Foster, C. (2004). Monitoring exercise intensity during resistance training using the session RPE scale. J Strength Cond Res, 18(2), 353-358. doi:10.1519/r-13113.1
13. Dungan, C. M., Peck, B. D., Walton, R. G., Huang, Z., Bamman, M. M., Kern, P. A., & Peterson, C. A. (2020). In vivo analysis of γH2AX+ cells in skeletal muscle from aged and obese humans. Faseb j, 34(5), 7018-7035. doi:10.1096/fj.202000111RR
14. Erben, R. G., Odörfer, K. I., Siebenhütter, M., Weber, K., & Rohleder, S. (2008). Histological assessment of cellular half-life in tissues in vivo. Histochem Cell Biol, 130(5), 1041-1046. doi:10.1007/s00418-008-0470-3
15. Fan Z H, Isobe K, Kiuchi K, Nakashima I (1995). Enhancement of nitric oxide production from activated macrophages by a purified form of ginsenoside (Rg1). Am J Chin Med. 1995; 23:279-87. doi.org/10.1142/50192415X9500033X PMID:8571924
16. Goswami, R., & Awasthi, A. (2020). T cell differentiation and function in tissue inflammation. Frontiers Immunol, 11, 289-289. doi:10.3389/fimmu.2020.00289
17. Guo X, Zhang J, Liu M, Zhao G C (2019). Protective effect of ginsenoside Rg1 on attenuating anti-GBM glomerular nephritis by activating NRF2 signalling. Artif Cells Nanomed Biotechnol. 2019; 47:2972-79. doi.org/10.1080/21691401.2019.1640712 PMID:31322005
18. Hashimoto, K., Kouno, T., Ikawa, T., Hayatsu, N., Miyajima, Y., Yabukami, H., Carninci, P. (2019). Single-cell transcriptomics reveals expansion of cytotoxic CD4 T cells in supercentenarians. Proc Natl Acad Sci USA, 116(48), 24242-24251. doi:10.1073/pnas.1907883116
19. Helms, E. R., Cronin, J., Storey, A., & Zourdos, M. C. (2016). Application of the repetitions in reserve-based rating of perceived exertion scale for resistance training. Strength Cond J, 38(4), 42-49. doi:10.1519/ssc.0000000000000218
20. Hou C W, Lee S D, Kao C L, Cheng I S, Lin Y N, Chuang S J, Chen C Y, Ivy J L, Huang C Y, Kuo C H (2015). Improved inflammatory balance of human skeletal muscle during exercise after supplementations of the ginseng-based steroid Rg1. PLoS One. 2015; 10:e0116387. doi.org/10.1371/journal.pone.0116387 PMID:25617625
21. Janzen, V., Forkert, R., Fleming, H. E., Saito, Y., Waring, M. T., Dombkowski, D. M., . . . Scadden, D. T. (2006). Stem-cell ageing modified by the cyclin-dependent kinase inhibitor p16INK4a. Nature, 443(7110), 421-426. doi:10.1038/nature05159
22. Joo S S, Won T J, Lee D I (2005). Reciprocal activity of ginsenosides in the production of proinflammatory repertoire, and their potential roles in neuroprotection in vivo. Planta Med. 2005; 71:476-81. doi.org/10.1055/s-2005-864145 PMID:15931589
23. Kay M M (1975). Mechanism of removal of senescent cells by human macrophages in situ. Proc Natl Acad Sci USA. 1975; 72:3521-25. doi.org/10.1073/pnas.72.9.3521 PMID:1059140
24. Justice, J. N., Gregory, H., Tchkonia, T., LeBrasseur, N. K., Kirkland, J. L., Kritchevsky, S. B., & Nicklas, B. J. (2018). Cellular senescence biomarker p16INK4a+ cell burden in thigh adipose is associated with poor physical function in older women. J Gerontol, 73(7), 939-945. doi:10.1093/gerona/glx134
25. Lasry, A., & Ben-Neriah, Y. (2015). Senescence-associated inflammatory responses: aging and cancer perspectives. Trends Immunol, 36(4), 217-228. doi:10.1016/j.it.2015.02.009
26. Lee, E. J., Ko, E., Lee, J., Rho, S., Ko, S., Shin, M. K., Bae, H. (2004). Ginsenoside Rg1 enhances CD4(+) T-cell activities and modulates Th1/Th2 differentiation. Int Immunopharmacol, 4(2), 235-244. doi:10.1016/j.intimp.2003.12.007
27. Liu, Y., Sanoff, H. K., Cho, H., Burd, C. E., Torrice, C., Ibrahim, J. G., Sharpless, N. E. (2009). Expression of p16(INK4a) in peripheral blood T-cells is a biomarker of human aging. Aging Cell, 8(4), 439-448. doi:10.1111/j.1474-9726.2009.00489.x
28. Malm, C., Nyberg, P., Engstrom, M., Sjodin, B., Lenkei, R., Ekblom, B., & Lundberg, I. (2000). Immunological changes in human skeletal muscle and blood after eccentric exercise and multiple biopsies. J Physiol, 529 Pt/(Pt 1), 243-262. doi:10.1111/j.1469-7793.2000.00243.x
29. Prata L G, Ovsyannikova I G, Tchkonia T, Kirkland J L (2018). Senescent cell clearance by the immune system: emerging therapeutic opportunities. Semin Immunol. 2018; 40:101275. doi.org/10.1016/j.smim.2019.04.003 PMID:31088710
30. Ressler, S., Bartkova, J., Niederegger, H., Bartek, J., Scharffetter-Kochanek, K., Jansen-Dürr, P., & Wlaschek, M. (2006). p16INK4A is a robust in vivo biomarker of cellular aging in human skin. Aging Cell, 5(5), 379-389. doi: 10.1111/j 0.1474-9726.2006.00231.x
31. Ribeiro, F., Ribeiro, I. P., Gonsalves, A. C., Alves, A. J., Melo, E., Fernandes, R., Oliveira, J. (2017). Effects of resistance exercise on endothelial progenitor cell mobilization in women. Sci Rep, 7(1), 17880. doi:10.1038/s41598-017-18156-6
32. Rochefort G Y, Vaudin P, Bonnet N, Pages J C, Domenech J, Charbord P, Eder V (2005). Influence of hypoxia on the domiciliation of mesenchymal stem cells after infusion into rats: possibilities of targeting pulmonary artery remodeling via cells therapies? Respir Res. 2005; 6:125. doi.org/10.1186/1465-9921-6-125 PMID: 16253136
33. Sarkar-Agrawal P, Vergilis I, Sharpless N E, DePinho R A, Rünger T M (2004). Impaired processing of DNA photoproducts and ultraviolet hypermutability with loss of p16INK4a or p19ARF. J Natl Cancer Inst. 2004; 96:1790-93. doi.org/10.1093/jnci/djh307 PMID:15572761
34. Sagiv A, Biran A, Yon M, Simon J, Lowe S W, Krizhanovsky V (2013). Granule exocytosis mediates immune surveillance of senescent cells. Oncogene. 2013; 32:1971-77. doi.org/10.1038/onc.2012.206 PMID:22751116
35. Serrano M (2014). Senescence helps regeneration. Dev Cell. 2014; 31:671-72. doi.org/10.1016/j.devce1.2014.12.007 PMID:25535913
36. Sharpless, N. E. (2004). Ink4a/Arf links senescence and aging. Exp Gerontol, 39(11), 1751-1759. doi:10.1016/j.exger.2004.06.025
37. Spalding, K. L., Bhardwaj, R. D., Buchholz, B. A., Druid, H., & Frisén, J. (2005). Retrospective birth dating of cells in humans. Cell, 122(1), 133-143. doi:10.1016/j.cell.2005.04.028
38. Sriwatanakul, K., Kelvie, W., & Lasagna, L. (1982). The quantification of pain: an analysis of words used to describe pain and analgesia in clinical trials. Clin Pharmacol Ther, 32(2), 143-148. doi:10.1038/clpt.1982.139
39. Storer, M., Mas, A., Robert-Moreno, A., Pecoraro, M., Ortells, M. C., Di Giacomo, V., Keyes, W. M. (2013). Senescence is a developmental mechanism that contributes to embryonic growth and patterning. Cell, 155(5), 1119-1130. doi:10.1016/j.ce11.2013.10.041
40. Tamaki, T., Akatsuka, A., Ando, K., Nakamura, Y., Matsuzawa, H., Hotta, T., Edgerton, V. R. (2002). Identification of myogenic-endothelial progenitor cells in the interstitial spaces of skeletal muscle. J Cell Biol, 157(4), 571-577. doi:10.1083/jcb.200112106
41. Tidball, J. G. (2017). Regulation of muscle growth and regeneration by the immune system. Nat Rev Immunol, 17, 165. doi:10.1038/nri.2016.150
42. Tryfidou D V, McClean C, Nikolaidis M G, Davison G W. DNA damage following acute aerobic exercise: a systematic review and meta-analysis. Sports Med. 2020; 50:103-27. doi.org/10.1007/s40279-019-01181-y
43. Tsai H H, Lin C P, Lin Y H, Hsu C C, Wang J S (2016). High-intensity interval training enhances mobilization/functionality of endothelial progenitor cells and depressed shedding of vascular endothelial cells undergoing hypoxia. Eur J Appl Physiol. 2016; 116:2375-88. doi.org/10.1007/s00421-016-3490-z
44. Urbich C, Dimmeler S (2004). Endothelial progenitor cells: characterization and role in vascular biology. Circ Res. 2004; 95:343-53. doi.org/10.1161/01.RES.0000137877.89448.78 PMID:15321944
45. Vasa, M., Fichtlscherer, S.; Aicher, A.; Adler, K.; Urbich, C.; Martin, H.; Zeiher, A. M.; Dimmeler, S (2001). Number and migratory activity of circulating endothelial progenitor cells inversely correlate with risk factors for coronary artery disease. Circ Res 2001, 89, E1-7, doi:10.1161/hh1301.093953
46. Wang Y, Wehling-Henricks M, Samengo G, Tidball J G (2015). Increases of M2a macrophages and fibrosis in aging muscle are influenced by bone marrow aging and negatively regulated by muscle-derived nitric oxide. Aging Cell. 2015; 14:678-88. doi.org/10.1111/ace1.12350 PMID:26009878
47. Wu, J., Saovieng, S., Cheng, I. S., Jensen, J., Jean, W.-H., Alkhatib, A., Kuo, C.-H. (2019). Satellite cells depletion in exercising human skeletal muscle is restored by ginseng component Rg1 supplementation. J Func Foods, 58, 27-33. doi.org/10.1016/j.jgr.2018.06.002 PMID:31695564
48. Yang, C., Jiao, Y., Wei, B., Yang, Z., Wu, J. F., Jensen, J., Kuo, C. H. (2018). Aged cells in human skeletal muscle after resistance exercise. Aging (Albany N.Y.), 10(6), 1356-1365. doi:10.18632/aging.101472
49. Zampetaki, A., Kirton, J. P., & Xu, Q. (2008). Vascular repair by endothelial progenitor cells. Cardiovasc Res, 78(3), 413-421. doi:10.1093/cvdcvn081
50. Zheng B, Cao B, Crisan M, Sun B, Li G, Logar A, Yap S, Pollett J B, Drowley L, Cassino T, Gharaibeh B, Deasy B M, Huard J, Péault B (2007). Prospective identification of myogenic endothelial cells in human skeletal muscle. Nat Biotechnol. 2007; 25:1025-34. doi.org/10.1038/nbt1334

What is claimed is:

1. A method for rejuvenation of muscle cells in a subject, which comprises administering to said subject an Rg1 supplement after his/her completing of an acute bout of squat exercise, wherein the supplement Rg1 comprises, consisting essentially of, or consisting of (1) Panax ginseng extract or Panax notoginseng extract and (2) Rosa roxburghii extract at an amount to reverse the aging of muscle cells in said subject, and is standardized to contain 30% to 40% of a total saponin, 0.6% to 2.0% of Vitamin C, and 2.0% to 4.0% of polyphenols, and a ginsenoside Rg1 as one indicator component ranging from 5 mg to 50 mg for one serving.

2. The method of claim 1, wherein the muscle cells are skeletal muscle cells.

3. The method of claim 1, wherein the Rg1 supplement enhances a reduction of stem cell aging in exercised skeletal muscle cells.

4. The method of claim 2, wherein the Rg1 supplement enhances a reduction of stem cell aging in exercised skeletal muscle cells.


SEQUENCE LISTING (12 sequences)

SEQ ID NO: 1 - DNA, 24 bp, Artificial Sequence
  primer
acaggattgacagattgatagctc

SEQ ID NO: 2 - DNA, 21 bp, Artificial Sequence
  primer
tcgctccaccaactaagaacg

SEQ ID NO: 3 - DNA, 24 bp, Artificial Sequence
  probe
tgcaccaccacccacggaatcgag

SEQ ID NO: 4 - DNA, 18 bp, Artificial Sequence
  primer
cttccctgtgaaaacaag

SEQ ID NO: 5 - DNA, 21 bp, Artificial Sequence
  primer
agacctctaatttatgtccta

SEQ ID NO: 6 - DNA, 20 bp, Artificial Sequence
  probe
agtcgccaccctgatgtctc

SEQ ID NO: 7 - DNA, 24 bp, Artificial Sequence
  primer
tgagatcgagtacatcttcaagcc

SEQ ID NO: 8 - DNA, 21 bp, Artificial Sequence
  primer
ggccttggtgaggtttgatcc

SEQ ID NO: 9 - DNA, 24 bp, Artificial Sequence
  probe
cctgtgtgcccctgatgcgatgcg

SEQ ID NO: 10 - DNA, 22 bp, Artificial Sequence
  primer
cgaggaatatcagcacgagagg

SEQ ID NO: 11 - DNA, 22 bp, Artificial Sequence
  primer
cataaatcacacggcgctcttc

SEQ ID NO: 12 - DNA, 25 bp, Artificial Sequence
  probe
tgccttctgcctctgcctctccctc